SEQUENCE LISTING (31 sequences)

SEQ ID NO: 1 - DNA, 2451 bp, Acinetobacter baylyi
tctttgactttgaaataaaaaagcgccatattcggcgctttttttatcatgattgctggcgatagtgtttaggagattgttcaaattgacgtttaaatgcctgactaaaagctgtttctgaggagtatccgactttattggcaatttgctgaatatttaactgactggtttttaataactgtacggccagacgaaggcgatgttgttgaagataagcgagaggggtctcatgaacaatactttgaaaaagtgtggcaaatttagaccgtgacatgcaacattgatctgccaaactctctacagtccaaggtttttcggggtaagcatgaatagctgcgagcgcattagataattcgggatgcatgagtgcagttaaccagcttttggggtcatcaagctgggcaatatgatcgcggacacattctataaataggatgctcatcaagtggtcaaatattttatctcgtcctggttgaatacgctgggtttcaagtgccacaaaatataaaccgacgcgtaaccactctgggccagttgagctcattgcgtgatgaatatgtatcagtgatggtaaagcattgagtaaagggctacccatatgcatatcgatatggctccgcacagtaaaaattaaaccatgatccgaagatgttgtgcctaactcaatggcatcattgcggtgtccatcaaaaaaagactgaatgttggaacaatcaattagtttgttctgagcatcatttgagacaaaatgagcttgcccagaaggaataagcacaatatctcccgaataagcggtttgtgtcgtaccatcctgtaatgtgaaatgagcagaacccattaaaataatgtgagcaatcaaagcatcttgggcaagcatatgaaatgcccattctccttgagtttttacatagagatattcagatttatttaaatgaatatcatcaaaaattttactaagtgcatccatagcgcgttaaaaattcttatttgagcaagagtatagaatgagattgtagcttatacatgtgattttaggacattgacatggttgaaaatgccaaagactttgacataggttttttggacgatcacaactgtttttaatatataagatggatgattatgagcagtatgaatagaggataacaagaaaggattcactatgaatgcacctgtacatgtcgatcaaaactttgaagaagtgatcaatgctgcaagatcaatgcgtgaaattgatcgtaaacgttacttatggatgattagtcctgcattaccagtcattgggatcgggattttggcaggttatcagttttcaccccgtccaatcaaaaaaatatttgcgctaggtggccctattgttctacacattattattccggtgattgatacgattattggaaaagatgccagcaatccaacgtctgaagaaatcaagcagcttgaaaatgatccttattacgcacgtttggtgaaaagcttcattccattacagtatattgccaatgtttatgcctgttatttggtgagccgtaaaaagacatcttttattgataaaattcttttaggcatttcgatgggtgcaatcaatggtattgcagtgaataccgcgcatgaattgagccataaagcagatcgactcgatcatatcctgtctcatttggcgctcgttcctacaggatacaatcactttcgtatagaacatccttacggacatcacaagcgtgcagcgactcctgaagatccagcttcgtcgcaaatgggtgaaacattttatgagttttggccaagaacggtctttggttcattaaaatctgcaatcgaaattgagacacatcgtctgaaacgtaaaggcaaaaagttttggtcaaaagataatgaactattacagggctgggggatgagtgctgcttttcatagttcgataattgcgatatttggtaaaggaacgattccttatctggttacgcaagctttttacgggattagtttatttgagatcattaattatatcgagcattatggtctaaaacgccagaaaagagcggatggcaattacgaacgtaccatgccagagcatagctggaataataacaatatcgtgacgaatctgtttttataccagttacaacgacattcagatcatcacgcttatccgacgcgtccatttcaggcattgcgtcattttgatgaagcacctgagttaccaagtggttatgccagtatgttgttgcctgctatgattccgccactttggtttaaaatgatggataaacgagtatttgagcattataaagaggatttgaccaaagccaatatctatccaaaacgtcgtgccaagatattggccaagtttggtttgactgacccgaatatagaaaacggcaagtaatcagaccttattttaaaagagagtctaagtgactctctttttattagattaagccaaagctt

SEQ ID NO: 2 - DNA, 1264 bp, Acinetobacter baylyi
tctttgactttgaaataaaaaagcgccatattcggcgctttttttatcatgattgctggcgatagtgtttaggagattgttcaaattgacgtttaaatgcctgactaaaagctgtttctgaggagtatccgactttattggcaatttgctgaatatttaactgactggtttttaataactgtacggccagacgaaggcgatgttgttgaagataagcgagaggggtctcatgaacaatactttgaaaaagtgtggcaaatttagaccgtgacatgcaacattgatctgccaaactctctacagtccaaggtttttcggggtaagcatgaatagctgcgagcgcattagataattcgggatgcatgagtgcagttaaccagcttttggggtcatcaagctgggcaatatgatcgcggacacattctataaataggatgctcatcaagtggtcaaatattttatctcgtcctggttgaatacgctgggtttcaagtgccacaaaatataaaccgacgcgtaaccactctgggccagttgagctcattgcgtgatgaatatgtatcagtgatggtaaagcattgagtaaagggctacccatatgcatatcgatatggctccgcacagtaaaaattaaaccatgatccgaagatgttgtgcctaactcaatggcatcattgcggtgtccatcaaaaaaagactgaatgttggaacaatcaattagtttgttctgagcatcatttgagacaaaatgagcttgcccagaaggaataagcacaatatctcccgaataagcggtttgtgtcgtaccatcctgtaatgtgaaatgagcagaacccattaaaataatgtgagcaatcaaagcatcttgggcaagcatatgaaatgcccattctccttgagtttttacatagagatattcagatttatttaaatgaatatcatcaaaaattttactaagtgcatccatagcgcgttaaaaattcttatttgagcaagagtatagaatgagattgtagcttatacatgtgattttaggacattgacatggttgaaaatgccaaagactttgacataggttttttggacgatcacaactgtttttaatatataagatggatgattatgagcagtatgaatagaggataacaagaaaggattcactatgaatgcacctgtacatgtcgatcaaaactttgaagaagtgatcaatgctgcaagatcaatgcgtgaaattgatcgtaaacgttacttatggatgattagt

SEQ ID NO: 3 - DNA, 1210 bp, Acinetobacter baylyi
tctttgactttgaaataaaaaagcgccatattcggcgctttttttatcatgattgctggcgatagtgtttaggagattgttcaaattgacgtttaaatgcctgactaaaagctgtttctgaggagtatccgactttattggcaatttgctgaatatttaactgactggtttttaataactgtacggccagacgaaggcgatgttgttgaagataagcgagaggggtctcatgaacaatactttgaaaaagtgtggcaaatttagaccgtgacatgcaacattgatctgccaaactctctacagtccaaggtttttcggggtaagcatgaatagctgcgagcgcattagataattcgggatgcatgagtgcagttaaccagcttttggggtcatcaagctgggcaatatgatcgcggacacattctataaataggatgctcatcaagtggtcaaatattttatctcgtcctggttgaatacgctgggtttcaagtgccacaaaatataaaccgacgcgtaaccactctgggccagttgagctcattgcgtgatgaatatgtatcagtgatggtaaagcattgagtaaagggctacccatatgcatatcgatatggctccgcacagtaaaaattaaaccatgatccgaagatgttgtgcctaactcaatggcatcattgcggtgtccatcaaaaaaagactgaatgttggaacaatcaattagtttgttctgagcatcatttgagacaaaatgagcttgcccagaaggaataagcacaatatctcccgaataagcggtttgtgtcgtaccatcctgtaatgtgaaatgagcagaacccattaaaataatgtgagcaatcaaagcatcttgggcaagcatatgaaatgcccattctccttgagtttttacatagagatattcagatttatttaaatgaatatcatcaaaaattttactaagtgcatccatagcgcgttaaaaattcttatttgagcaagagtatagaatgagattgtagcttatacatgtgattttaggacattgacatggttgaaaatgccaaagactttgacataggttttttggacgatcacaactgtttttaatatataagatggatgattatgagcagtatgaatagaggataacaagaaaggattcactatgaatgcacctgtacatgtcgatcaaaactttgaagaagtgatcaat

SEQ ID NO: 4 - DNA, 1165 bp, Acinetobacter baylyi
tctttgactttgaaataaaaaagcgccatattcggcgctttttttatcatgattgctggcgatagtgtttaggagattgttcaaattgacgtttaaatgcctgactaaaagctgtttctgaggagtatccgactttattggcaatttgctgaatatttaactgactggtttttaataactgtacggccagacgaaggcgatgttgttgaagataagcgagaggggtctcatgaacaatactttgaaaaagtgtggcaaatttagaccgtgacatgcaacattgatctgccaaactctctacagtccaaggtttttcggggtaagcatgaatagctgcgagcgcattagataattcgggatgcatgagtgcagttaaccagcttttggggtcatcaagctgggcaatatgatcgcggacacattctataaataggatgctcatcaagtggtcaaatattttatctcgtcctggttgaatacgctgggtttcaagtgccacaaaatataaaccgacgcgtaaccactctgggccagttgagctcattgcgtgatgaatatgtatcagtgatggtaaagcattgagtaaagggctacccatatgcatatcgatatggctccgcacagtaaaaattaaaccatgatccgaagatgttgtgcctaactcaatggcatcattgcggtgtccatcaaaaaaagactgaatgttggaacaatcaattagtttgttctgagcatcatttgagacaaaatgagcttgcccagaaggaataagcacaatatctcccgaataagcggtttgtgtcgtaccatcctgtaatgtgaaatgagcagaacccattaaaataatgtgagcaatcaaagcatcttgggcaagcatatgaaatgcccattctccttgagtttttacatagagatattcagatttatttaaatgaatatcatcaaaaattttactaagtgcatccatagcgcgttaaaaattcttatttgagcaagagtatagaatgagattgtagcttatacatgtgattttaggacattgacatggttgaaaatgccaaagactttgacataggttttttggacgatcacaactgtttttaatatataagatggatgattatgagcagtatgaatagaggataacaagaaaggattcactatg

SEQ ID NO: 5 - DNA, 921 bp, Acinetobacter baylyi
ttagctctgctgacgatagtgctttggagactgttcgaactgacgtttgaaggcctggctgaaggcagtctcggaggagtagcccactttgttagcgatctgctgaatgttcagctgggaggttttcagcagctgaacagccagacgcaggcgatgctgctgcaggtaagccagcggagtttcgtgtacgatggactgaaacagggtggcgaatttagaacggctcatacaacactggtcagccaggctttccacagtccacggcttttctgggtaagcgtggatagccgccagtgcgttgctcagttccgggtgcatcagggcggtcagccaggatttaggatcgtccagctgcgcaatgtggtcacgtacgcattcgatgaacagaatagacatcaggtggtcaaagatcttatcacgacccggttggatacgttgagtctccagcgcgacaaagtacaggcctacacgcagccattccgggccagtagagctcatagcatgatggatgtggatcagggacggcagcgcgttcagcagcggggaacccatgtgcatgtcgatgtgagaacgtacggtaaaaatcagaccgtggtcggagctggtggtgcccagttcaatcgcgtcgttgcggtggccatcgaagaagctctggatgttgctacagtcgatcagtttattctgagcatcattggaaacgaaatgggcctgaccagacgggatcagtacaatatcaccggagtacgcggtctgggtggtaccatcctgcagggtgaagtgcgcggaacccatcaggataatatgagcgatcagggcatcctgtgccagcatgtgaaatgcccactcgccctgggtttttacatacagatattcagatttgttcaggtgaatgtcatcgaagattttagacagcgcatccat

SEQ ID NO: 6 - DNA, 2528 bp, Acinetobacter sp.
ttcctcgaactgattaaagacgatgaagattacatgagcaccattctgccaattggtgcaggcttatgtatggtggtgttgaagtgaattgattttactttgatgatgtagtcgatatgcttgagggctacagccaatcactttattaaatgcacgggtgaaattggcaggattagaataacccagctcataggcaatatgggtaatggtaaagctagaatttactaataactcaatcgcacgttcagtgctgatcagctgttttaacgcttgaaattcaacgccttgttgctgtaaatagcgttgcaaggttttggtggaaatatttaagactttggcacattccatgagggttgggacttgattggcttgtcgcaacatcatgctgacccattccacaatttctccttgatgatagatcttctggatttgttcctgacagcgttgttcaactatttttaaactatgtgcatctgccaaaggtaaggggagtgccaattgctgctgatcaatcaccacgcggatacctgcaaaccaggtatagttaaaatggaaattggctttcaccaaggataaataacgttcttgatagctcggctctggaacactcaaatagacttggtagcgttgtaattgctggcctgccaattctaataggctgtagtagaacgctacggcaatcgcttcaatatgaaaggtcaaacattgcttattcatctgcaaaattggttcaaataacagttcaactttttgtgagcctgctggaaactgaatgctgagcttaaaactgggcataatcagtctaaaatattgtgcaatcagcctaagcgcatgttccagattaggactggtcatgagtgcatagccgacgagactatgtgagctgagctttatatttttacccagttcaaaggccaagtcttgggtattgggtaggcttaaaccaatttctatgaattgttcaatttgttgaatcgataataattctgtttttgccagttcttgctgcacagtcatcaacaaatcattatcgtattgatgcgtaccgattaaaatttcaatcagtcgtaaataataacgtgatggaataacagggatctgctgcttttgcacaagaaaaactcattaaagtcttaaaatgataatagcaagtcttaaaatgataatatagagggatagtgaaaggacacaatcaattcaagcaaaacattttgtgtttgattgaggaatttctaatgaatatgcatactcaactggatgtcgagcaagcgacaacaacatttaaggataagaaacgtcatttatggctattgggtttggctgtaccgaccattgcaatgggtggtttggcgggctatcaatttggccctaaaaaaaccaaaaagttcttcgcttcatttgggccattatttattcatggcgttattccaacattagataaattgattggtgaagacaccgagaacccgccattagatgcaatcgcagatttagaagctgatccgtattacgctcgtattgttaaactttttattcctttacaatacgcgaccaatatttatgggacctatttagccagtcgtaaagacacaccagtcgcagatcaagtgttattgggaactttagtaggcatggtaaatggaattgccattaatactgcacatgaactgagccataagagcggacgtttagagcattatctttcgcatttggctttagctccgtcaggttataaccatttccgaattgaacacccgtatggacatcatcgtcgcgttgcaacacctgaagatccagcatcgtcacaatttggcgaaagcttttggaagtttttaccacgtaccgtgattggtagttttaaatcagcaatcgaaattgagaaaaaccgtttagaacgtaagaaactgcctttcttctgtaaggaaaatgagctgattcatggctgggcgatgtctgctgtatatcatgctgccatgttgagcaaatttggtatgcgctcagtcccattccaagccacacaagcggcttatgcaattactttatttgaatcagtaaattatattgaacattatggtttaaagcgtgcaaagaaagaaaatggccaatatgaacgtactttgccagagcatagctggaacaataataatgtggtgaccaatttgttcttgtatcaattacaacgtcattcggatcatcacgccaatccgactcgaagcttccaaactttgcgtcattttgaagatgcgccacaattacctgctggttatggtgccatgattctaccagcatttattccatcttggtggtcaaaaattatggatgatcgagtggtgcaacactataaaggtaatttggacagaataaatgtccatcctgaagccaaacagaagatgttagagaaatatgctgaacagcaagatgctgcttaaagctgaagtgagatacaacgaaaatcagcccaatcgggctgattttttgttttttaagctgaaatacagagtcgtttaaaatgcagatcaaattctaga

SEQ ID NO: 7 - DNA, 1343 bp, Acinetobacter sp.
ttcctcgaactgattaaagacgatgaagattacatgagcaccattctgccaattggtgcaggcttatgtatggtggtgttgaagtgaattgattttactttgatgatgtagtcgatatgcttgagggctacagccaatcactttattaaatgcacgggtgaaattggcaggattagaataacccagctcataggcaatatgggtaatggtaaagctagaatttactaataactcaatcgcacgttcagtgctgatcagctgttttaacgcttgaaattcaacgccttgttgctgtaaatagcgttgcaaggttttggtggaaatatttaagactttggcacattccatgagggttgggacttgattggcttgtcgcaacatcatgctgacccattccacaatttctccttgatgatagatcttctggatttgttcctgacagcgttgttcaactatttttaaactatgtgcatctgccaaaggtaaggggagtgccaattgctgctgatcaatcaccacgcggatacctgcaaaccaggtatagttaaaatggaaattggctttcaccaaggataaataacgttcttgatagctcggctctggaacactcaaatagacttggtagcgttgtaattgctggcctgccaattctaataggctgtagtagaacgctacggcaatcgcttcaatatgaaaggtcaaacattgcttattcatctgcaaaattggttcaaataacagttcaactttttgtgagcctgctggaaactgaatgctgagcttaaaactgggcataatcagtctaaaatattgtgcaatcagcctaagcgcatgttccagattaggactggtcatgagtgcatagccgacgagactatgtgagctgagctttatatttttacccagttcaaaggccaagtcttgggtattgggtaggcttaaaccaatttctatgaattgttcaatttgttgaatcgataataattctgtttttgccagttcttgctgcacagtcatcaacaaatcattatcgtattgatgcgtaccgattaaaatttcaatcagtcgtaaataataacgtgatggaataacagggatctgctgcttttgcacaagaaaaactcattaaagtcttaaaatgataatagcaagtcttaaaatgataatatagagggatagtgaaaggacacaatcaattcaagcaaaacattttgtgtttgattgaggaatttctaatgaatatgcatactcaactggatgtcgagcaagcgacaacaacatttaaggataagaaacgtcatttatggctattgggtttggctgtaccgaccattgcaatgggt

SEQ ID NO: 8 - DNA, 1289 bp, Acinetobacter sp.
ttcctcgaactgattaaagacgatgaagattacatgagcaccattctgccaattggtgcaggcttatgtatggtggtgttgaagtgaattgattttactttgatgatgtagtcgatatgcttgagggctacagccaatcactttattaaatgcacgggtgaaattggcaggattagaataacccagctcataggcaatatgggtaatggtaaagctagaatttactaataactcaatcgcacgttcagtgctgatcagctgttttaacgcttgaaattcaacgccttgttgctgtaaatagcgttgcaaggttttggtggaaatatttaagactttggcacattccatgagggttgggacttgattggcttgtcgcaacatcatgctgacccattccacaatttctccttgatgatagatcttctggatttgttcctgacagcgttgttcaactatttttaaactatgtgcatctgccaaaggtaaggggagtgccaattgctgctgatcaatcaccacgcggatacctgcaaaccaggtatagttaaaatggaaattggctttcaccaaggataaataacgttcttgatagctcggctctggaacactcaaatagacttggtagcgttgtaattgctggcctgccaattctaataggctgtagtagaacgctacggcaatcgcttcaatatgaaaggtcaaacattgcttattcatctgcaaaattggttcaaataacagttcaactttttgtgagcctgctggaaactgaatgctgagcttaaaactgggcataatcagtctaaaatattgtgcaatcagcctaagcgcatgttccagattaggactggtcatgagtgcatagccgacgagactatgtgagctgagctttatatttttacccagttcaaaggccaagtcttgggtattgggtaggcttaaaccaatttctatgaattgttcaatttgttgaatcgataataattctgtttttgccagttcttgctgcacagtcatcaacaaatcattatcgtattgatgcgtaccgattaaaatttcaatcagtcgtaaataataacgtgatggaataacagggatctgctgcttttgcacaagaaaaactcattaaagtcttaaaatgataatagcaagtcttaaaatgataatatagagggatagtgaaaggacacaatcaattcaagcaaaacattttgtgtttgattgaggaatttctaatgaatatgcatactcaactggatgtcgagcaagcgacaacaacatttaaggat

SEQ ID NO: 9 - DNA, 1244 bp, Acinetobacter sp.
ttcctcgaactgattaaagacgatgaagattacatgagcaccattctgccaattggtgcaggcttatgtatggtggtgttgaagtgaattgattttactttgatgatgtagtcgatatgcttgagggctacagccaatcactttattaaatgcacgggtgaaattggcaggattagaataacccagctcataggcaatatgggtaatggtaaagctagaatttactaataactcaatcgcacgttcagtgctgatcagctgttttaacgcttgaaattcaacgccttgttgctgtaaatagcgttgcaaggttttggtggaaatatttaagactttggcacattccatgagggttgggacttgattggcttgtcgcaacatcatgctgacccattccacaatttctccttgatgatagatcttctggatttgttcctgacagcgttgttcaactatttttaaactatgtgcatctgccaaaggtaaggggagtgccaattgctgctgatcaatcaccacgcggatacctgcaaaccaggtatagttaaaatggaaattggctttcaccaaggataaataacgttcttgatagctcggctctggaacactcaaatagacttggtagcgttgtaattgctggcctgccaattctaataggctgtagtagaacgctacggcaatcgcttcaatatgaaaggtcaaacattgcttattcatctgcaaaattggttcaaataacagttcaactttttgtgagcctgctggaaactgaatgctgagcttaaaactgggcataatcagtctaaaatattgtgcaatcagcctaagcgcatgttccagattaggactggtcatgagtgcatagccgacgagactatgtgagctgagctttatatttttacccagttcaaaggccaagtcttgggtattgggtaggcttaaaccaatttctatgaattgttcaatttgttgaatcgataataattctgtttttgccagttcttgctgcacagtcatcaacaaatcattatcgtattgatgcgtaccgattaaaatttcaatcagtcgtaaataataacgtgatggaataacagggatctgctgcttttgcacaagaaaaactcattaaagtcttaaaatgataatagcaagtcttaaaatgataatatagagggatagtgaaaggacacaatcaattcaagcaaaacattttgtgtttgattgaggaatttctaatgaatatg

SEQ ID NO: 10 - DNA, 954 bp, Acinetobacter sp.
ttacgtgtgatggtgctgaagggaattgatctttgattgatggtgcaaacgatatgcctgaggactacaaccaataactttgttgaaggcccgtgtaaaattagctgggttagagtagccgagttcatacgcaatatgagtgatggtgaagctcgaattgaccagcagttctattgcgcgctccgtggaaattaattgtttaagggcctgaaattccactccctgttgctgcaggtaacgttgcaaggtctttgttgagatgttgagaactttcgcgcactccatcagagtcggtacctgattagcctggcgtaacatcatactcacccattcgactatttccccttggtgataaattttctggatttgctcctgacaacgctgttcaacaatcttcagagaatgtgcatcggcaaggggcaacggcagcgcgagttgctgttggtcgatcactactcgtataccagcaaaccacgtatagttgaagtgaaaatttgctttgaccaggcttaagtatctctcctgatacgaaggttctggaacggacaggtacacctgataccgttgaagctgttggccggccaattccagtaatgagtaatagaacgctacagcaattgcctcgatatgaaaggtgaggcactgtttgttcatctgcagaatcggttcgaaaagcaactcgaccttttgacttccggcgggaaactgtatagacagtttgaagctaggcatgattaagcgaaagtattgcgcaatcagacggagagcgtgttccagatttggcgatgtcataagtgcatacccaaccaaggaatgtgaacttaatttgatgttcttacccagttcgaaggccagatcctgagtattcgggagagaaaggccaatctctataaactgttcgatttgctgaatgctcaataactccgttttcgccagttcttgctgcacggtcat

SEQ ID NO: 11 - DNA, 2573 bp, Acinetobacter sp.
tcactgagcagaaaagaaaaaagcaccgcggtgtctttttttataactttaactttcttgctcaacaaactgctgacgatattgcttcggagagtgttcaaaggttcgcttaaatgcctgactaaacgcagtttctgaagagtagccaactttattggcgatttgttggactgaaaaattacttttacgcaaatattggcttgctaagcgcatgcgatgttgctgtaaataagcgaggggcgattcgccaatcacttcgtgaaatagactggcaaatttcgagcgtgacatgcagcattgttctgccaatgattcaacggtccaagcatcttcagggtgattgtgaattgcagctaagctattgctcagttcgggatgatttaaagcgctgagccaaccatgttgacttgagatttgttgaatatgatcgcgaatacatttaatgagtaagatattcacgagatgatcgataataatatcgcggcctgcctgaatattctgcgtttctaaggccagaaagtgtaagccgatttgcagccattcaggtgcattggtatctccatgttggagatggatgattggaggtagagcctgaataaacggacgagccataatcgtatcaatctggcaacgcacggtcaaaattagatttttctcagatgactgagtaccgaattcgatagcatcttctttatggccatcgaacagctttgaaatatcaacggcatcgaccagtttagtaatcgtattatctgcgcctgtatgtgcttttcctgacgggatcaccacaatttcacccgcatgtgcagttaaactattttgtgcgtcaatttgaataaaaacagaaccaactaaaacaatatgaacaattaaggcggtttgatcttggcagataaaactccattctccttgagttttcaagtagatatattcagtgtgatttaaatgaatatcagcaaatattttacttaaagcatccatatgattttttaacgatgtattcatttaaattatagagagctgcgccaataactcaatatgtgtttataggacaaaaacagggtggatgatgccaaagactttgacatagcttttttggacgcttgcaactgtcatgaatttcataattactcaaaatatacgtatatatacaaagacatggattcgatgatgaatgcgccagtaaatgttgagcaagaacttacgccagtagccattccaaagtcaaaaactgaattagatcgtaaacgttatttatgggccattagcccagctttacctgcaatcgggattggtattttagcaggttatcaatttgcaccacgtccattgaagaaaatcttcgctttaggtggtccaattgttctacatatcattattccaacgattgatacgattattggtaaagatgctaataatccaacggatgaagatattcgactacttgaaaaagacccttactattctcgattggttaaaagctttattcccctacaatatgcagcaaatatatatgcatgttatttaaccagtcgtaaagaaacatcattcattgataagatttttctagggatttcaatgggtgcgatcaacgggattgcgattaatactgcacatgagttgagtcataaacatgatcgtattgaccacattctgtcgcatttagcgcttgtgccaacgggttataaccatttccgaattgaacatccttatggccaccataaacgtgctgcaacacctgaagatccagcatcttcacaaatgggtgaaacattctatgagttctggccacgtaccgtgattggttcgtttaaatctgcaatagagattgaaaccaatcgtctaaaacgtaaaggtaaagaattctggtcaactgagaatgaactattacaaggttggggcatgagtgcggctttccatggttcaatggtcggtttgtttggtaagggcgttattccttatttggcaacacaggctttttatggcattagtttgtttgaaattattaactatatcgaacactatggtttaaaacgtcaaaaagacaagaatggtaagtatgagcgtaccatgccagaacatagctggaacaataataatattgtgaccaacttattcctgtatcaattgcaacgtcactcagatcatcatgcttatccgacacgtccgttccaggctttacgtcattttgatgaagcgcctgaattaccaagtggctatgcaagtatgctattacccgcattaattccaccactatggtcgaaaatgatggataagcgtgtatttgatcactacaaaggtgatttgaataaagccaatatttatccaaaacgtcgtgccaaaatctttaagaaatttggtgtggttgatcagtctttagaacaggttcagattgaagctgtgactgttgaataatcaaagctttcccaaagcagggcactgatgagtcatcatcagtgcctttttattttcttgtataaaaaaattaaaaaacaacggcttttatactttgttgctgtatatctataggtgagaacaaacaagaatcttgcaattgattttatgaggcgatg

SEQ ID NO: 12 - DNA, 1269 bp, Acinetobacter sp.
tcactgagcagaaaagaaaaaagcaccgcggtgtctttttttataactttaactttcttgctcaacaaactgctgacgatattgcttcggagagtgttcaaaggttcgcttaaatgcctgactaaacgcagtttctgaagagtagccaactttattggcgatttgttggactgaaaaattacttttacgcaaatattggcttgctaagcgcatgcgatgttgctgtaaataagcgaggggcgattcgccaatcacttcgtgaaatagactggcaaatttcgagcgtgacatgcagcattgttctgccaatgattcaacggtccaagcatcttcagggtgattgtgaattgcagctaagctattgctcagttcgggatgatttaaagcgctgagccaaccatgttgacttgagatttgttgaatatgatcgcgaatacatttaatgagtaagatattcacgagatgatcgataataatatcgcggcctgcctgaatattctgcgtttctaaggccagaaagtgtaagccgatttgcagccattcaggtgcattggtatctccatgttggagatggatgattggaggtagagcctgaataaacggacgagccataatcgtatcaatctggcaacgcacggtcaaaattagatttttctcagatgactgagtaccgaattcgatagcatcttctttatggccatcgaacagctttgaaatatcaacggcatcgaccagtttagtaatcgtattatctgcgcctgtatgtgcttttcctgacgggatcaccacaatttcacccgcatgtgcagttaaactattttgtgcgtcaatttgaataaaaacagaaccaactaaaacaatatgaacaattaaggcggtttgatcttggcagataaaactccattctccttgagttttcaagtagatatattcagtgtgatttaaatgaatatcagcaaatattttacttaaagcatccatatgattttttaacgatgtattcatttaaattatagagagctgcgccaataactcaatatgtgtttataggacaaaaacagggtggatgatgccaaagactttgacatagcttttttggacgcttgcaactgtcatgaatttcataattactcaaaatatacgtatatatacaaagacatggattcgatgatgaatgcgccagtaaatgttgagcaagaacttacgccagtagccattccaaagtcaaaaactgaattagatcgtaaacgttatttatgggccattagc

SEQ ID NO: 13 - DNA, 1215 bp, Acinetobacter sp.
tcactgagcagaaaagaaaaaagcaccgcggtgtctttttttataactttaactttcttgctcaacaaactgctgacgatattgcttcggagagtgttcaaaggttcgcttaaatgcctgactaaacgcagtttctgaagagtagccaactttattggcgatttgttggactgaaaaattacttttacgcaaatattggcttgctaagcgcatgcgatgttgctgtaaataagcgaggggcgattcgccaatcacttcgtgaaatagactggcaaatttcgagcgtgacatgcagcattgttctgccaatgattcaacggtccaagcatcttcagggtgattgtgaattgcagctaagctattgctcagttcgggatgatttaaagcgctgagccaaccatgttgacttgagatttgttgaatatgatcgcgaatacatttaatgagtaagatattcacgagatgatcgataataatatcgcggcctgcctgaatattctgcgtttctaaggccagaaagtgtaagccgatttgcagccattcaggtgcattggtatctccatgttggagatggatgattggaggtagagcctgaataaacggacgagccataatcgtatcaatctggcaacgcacggtcaaaattagatttttctcagatgactgagtaccgaattcgatagcatcttctttatggccatcgaacagctttgaaatatcaacggcatcgaccagtttagtaatcgtattatctgcgcctgtatgtgcttttcctgacgggatcaccacaatttcacccgcatgtgcagttaaactattttgtgcgtcaatttgaataaaaacagaaccaactaaaacaatatgaacaattaaggcggtttgatcttggcagataaaactccattctccttgagttttcaagtagatatattcagtgtgatttaaatgaatatcagcaaatattttacttaaagcatccatatgattttttaacgatgtattcatttaaattatagagagctgcgccaataactcaatatgtgtttataggacaaaaacagggtggatgatgccaaagactttgacatagcttttttggacgcttgcaactgtcatgaatttcataattactcaaaatatacgtatatatacaaagacatggattcgatgatgaatgcgccagtaaatgttgagcaagaacttacgccagtagcc

SEQ ID NO: 14 - DNA, 1170 bp, Acinetobacter sp.
tcactgagcagaaaagaaaaaagcaccgcggtgtctttttttataactttaactttcttgctcaacaaactgctgacgatattgcttcggagagtgttcaaaggttcgcttaaatgcctgactaaacgcagtttctgaagagtagccaactttattggcgatttgttggactgaaaaattacttttacgcaaatattggcttgctaagcgcatgcgatgttgctgtaaataagcgaggggcgattcgccaatcacttcgtgaaatagactggcaaatttcgagcgtgacatgcagcattgttctgccaatgattcaacggtccaagcatcttcagggtgattgtgaattgcagctaagctattgctcagttcgggatgatttaaagcgctgagccaaccatgttgacttgagatttgttgaatatgatcgcgaatacatttaatgagtaagatattcacgagatgatcgataataatatcgcggcctgcctgaatattctgcgtttctaaggccagaaagtgtaagccgatttgcagccattcaggtgcattggtatctccatgttggagatggatgattggaggtagagcctgaataaacggacgagccataatcgtatcaatctggcaacgcacggtcaaaattagatttttctcagatgactgagtaccgaattcgatagcatcttctttatggccatcgaacagctttgaaatatcaacggcatcgaccagtttagtaatcgtattatctgcgcctgtatgtgcttttcctgacgggatcaccacaatttcacccgcatgtgcagttaaactattttgtgcgtcaatttgaataaaaacagaaccaactaaaacaatatgaacaattaaggcggtttgatcttggcagataaaactccattctccttgagttttcaagtagatatattcagtgtgatttaaatgaatatcagcaaatattttacttaaagcatccatatgattttttaacgatgtattcatttaaattatagagagctgcgccaataactcaatatgtgtttataggacaaaaacagggtggatgatgccaaagactttgacatagcttttttggacgcttgcaactgtcatgaatttcataattactcaaaatatacgtatatatacaaagacatggattcgatg

SEQ ID NO: 15 - DNA, 35 bp, Artificial Sequence
  Description of Artificial Sequence Synthetic primer
ttttatttcaaagtcaaagactcgagaagcttggg

SEQ ID NO: 16 - DNA, 42 bp, Artificial Sequence
  Description of Artificial Sequence Synthetic primer
catgccatggtatatctccttaactaatcatccataagtaac

SEQ ID NO: 17 - DNA, 42 bp, Artificial Sequence
  Description of Artificial Sequence Synthetic primer
catgccatggtatatctccttaattgatcacttcttcaaagt

SEQ ID NO: 18 - DNA, 25 bp, Artificial Sequence
  Description of Artificial Sequence Synthetic primer
catgccatggtgaatcctttcttgt

SEQ ID NO: 19 - DNA, 35 bp, Artificial Sequence
  Description of Artificial Sequence Synthetic primer
tctttaatcagttcgaggaactcgagaagcttggg

SEQ ID NO: 20 - DNA, 45 bp, Artificial Sequence
  Description of Artificial Sequence Synthetic primer
gaattcccatatgtatatctccttaacccattgcaatggtcggta

SEQ ID NO: 21 - DNA, 45 bp, Artificial Sequence
  Description of Artificial Sequence Synthetic primer
gaattcccatatgtatatctccttaatccttaaatgttgttgtcg

SEQ ID NO: 22 - DNA, 27 bp, Artificial Sequence
  Description of Artificial Sequence Synthetic primer
gaattcccatatgcattagaaattcct

SEQ ID NO: 23 - DNA, 35 bp, Artificial Sequence
  Description of Artificial Sequence Synthetic primer
ttttcttttctgctcagtgactcgagaagcttggg

SEQ ID NO: 24 - DNA, 42 bp, Artificial Sequence
  Description of Artificial Sequence Synthetic primer
catgccatggtatatctccttagctaatggcccataaataac

SEQ ID NO: 25 - DNA, 42 bp, Artificial Sequence
  Description of Artificial Sequence Synthetic primer
catgccatggtatatctccttaggctactggcgtaagttctt

SEQ ID NO: 26 - DNA, 25 bp, Artificial Sequence
  Description of Artificial Sequence Synthetic primer
catgccatggaatccatgtctttgt

SEQ ID NO: 27 - protein, 306 aa, Acinetobacter baylyi
MDALSKIFDDIHLNKSEYLYVKTQGEWAFHMLAQDALIAHIILMGSAHFTLQDGTTQTAYSGDIVLIPSGQAHFVSNDAQNKLIDCSNIQSFFDGHRNDAIELGTTSSDHGLIFTVRSHIDMHMGSPLLNALPSLIHIHHAMSSTGPEWLRVGLYFVALETQRIQPGRDKIFDHLMSILFIECVRDHIAQLDDPKSWLTALMHPELSNALAAIHAYPEKPWTVESLADQCCMSRSKFATLFQSIVHETPLAYLQQHRLRLAVQLLKTSQLNIQQIANKVGYSSETAFSQAFKRQFEQSPKHYRQQS

SEQ ID NO: 28 - protein, 395 aa, Acinetobacter sp.
MHTQLDVEQATTTFKDKKRHLWLLGLAVPTIAMGGLAGYQFGPKKTKKFFASFGPLFIHGVIPTLDKLIGEDTENPPLDAIADLEADPYYARIVKLFIPLQYATNIYGTYLASRKDTPVADQVLLGTLVGMVNGIAINTAHELSHKSGRLEHYLSHLALAPSGYNHFRIEHPYGHHRRVATPEDPASSQFGESFWKFLPRTVIGSFKSAIEIEKNRLERKKLPFFCKENELIHGWAMSAVYHAAMLSKFGMRSVPFQATQAAYAITLFESVNYIEHYGLKRAKKENGQYERTLPEHSWNNNNVVTNLFLYQLQRHSDHHANPTRSFQTLRHFEDAPQLPAGYGAMILPAFIPSWWSKIMDDRVVQHYKGNLDRINVHPEAKQKMLEKYAEQQDAA

SEQ ID NO: 29 - protein, 318 aa, Acinetobacter sp.
MNTSLKNHMDALSKIFADIHLNHTEYIYLKTQGEWSFICQDQTALIVHIVLVGSVFIQIDAQNSLTAHAGEIVVIPSGKAHTGADNTITKLVDAVDISKLFDGHKEDAIEFGTQSSEKNLILTVRCQIDTIMARPFIQALPPIIHLQHGDTNAPEWLQIGLHFLALETQNIQAGRDIIIDHLVNILLIKCIRDHIQQISSQHGWLSALNHPELSNSLAAIHNHPEDAWTVESLAEQCCMSRSKFASLFHEVIGESPLAYLQQHRMRLASQYLRKSNFSVQQIANKVGYSSETAFSQAFKRTFEHSPKQYRQQFVEQES

SEQ ID NO: 30 - DNA, 23 bp, Artificial Sequence
  Description of Artificial Sequence Synthetic primer
catgccatggcgagcaaaggtga

SEQ ID NO: 31 - DNA, 23 bp, Artificial Sequence
  Description of Artificial Sequence Synthetic primer
acctctagactcgagaagctttt

(19) United States
(12) Patent Application Publication
(10) Pub. No.: US 2008/0293060 A1
(43) Pub. Date: Nov. 27, 2008

(54) Methods and Compositions for Identification of Hydrocarbon Response, Transport and Biosynthesis Genes

(75) Inventors: Andreas Schirmer, Fremont, CA (US); Zhihao Hu, Castro Valley, CA (US); Bernardo da Costa, San Francisco, CA (US)

Correspondence Address: FENWICK & WEST LLP, SILICON VALLEY CENTER, 801 CALIFORNIA STREET, MOUNTAIN VIEW, CA, 94041, US

(73) Assignee: LS9, Inc., South San Francisco, CA (US)

(21) Appl. No.: 12/108,278

(22) Filed: Apr. 23, 2008

Related U.S. Application Data
(60) Provisional application No. 60/913,449, filed on Apr. 23, 2007.

(51) Int. Cl. C12Q 1/68; C12N 1/21

(52) U.S. Cl. 435/6; 435/252.33; 435/252.3

(57) ABSTRACT

Disclosed is a method using an alkane response element (ARE) from, e.g., Acinetobacter spp. to (i) identify and clone hydrocarbon biosynthesis genes, (ii) identify and clone hydrocarbon transporter genes (iii) identify and clone hydrocarbon response genes. Screening cells were developed that expressed a transcriptional activator, e.g., alkR, and included a reporter gene, e.g., GFP operatively linked to an ARE promoter, e.g., the alkM promoter. The cells were transformed with libraries from organisms capable of hydrocarbon biosynthesis. Transformed cells that expressed the reporter gene harbored library-derived genes involved in one or more of the above-mentioned processes; and these genes were isolated from the cells using standard molecular biology techniques. Additional systems were designed wherein screening cells also expressed a gene identified in the original screen, e.g., an additional hydrocarbon pathway gene, e.g., an enhancer.

### RELATED APPLICATIONS

This application claims the benefit of U.S. Provisional Application No. 60/913,449, filed Apr. 23, 2007, the entire disclosure of which is hereby incorporated by reference in its entirety for all purposes. This application is related to U.S. Provisional Application Nos. 60/852,587, and 60/852,629 and 60/852,453, all filed on Oct. 17, 2006 which are hereby incorporated by reference in their entirety for all purposes.

### STATEMENT REGARDING FEDERALLY SPONSORED RESEARCH OR DEVELOPMENT

Not applicable.

### BACKGROUND OF THE INVENTION

1. Field of the Invention

The invention relates to screening methods and screening cells for identification of genes involved in the hydrocarbon biosynthesis, transport, and response, e.g., hydrocarbon pathway genes.

2. Description of the Related Art

Hydrocarbons are energy rich molecules with great commercial utility as fuels and chemical. The majority of hydrocarbons are currently derived from petrochemical sources, e.g., non-renewable sources. Recently efforts have been made to develop renewable sources of hydrocarbons. These efforts have focused on production of ethanol, butanol, biodiesel, and biohydrogen and the like from various renewable carbon sources, e.g., corn and cellulosic biomass. All of these sources of renewable energy have disadvantages related to expense, limited versatility, and unique infrastructure (e.g., distribution) requirements. It would be beneficial to develop a renewable hydrocarbon maintains the physical, chemical and energetic characteristics of current petroleum based products.

Numerous organisms, such as bacteria, algae, plants and some animals, can synthesize hydrocarbons, e.g., n-alkanes of various carbon chain lengths as previously described (Dennis, M. W. & Kolattukudy, P. E. (1991) Archives of biochemistry and biophysics 287, 268-275; Kunst, L. & Samuels, A. L. (2003) Progress in lipid research 42, 51-80; Tillman, J. A., Seybold, S. J., Jurenka, R. A., & Blomquist, G. J. (1999) Insect biochemistry and molecular biology 29, 481-514; Tornabene, T. G. (1982) Experientia 38.1-4, each of which is incorporated by reference). These alkane biosynthetic pathways are only poorly understood. On a genetic level, only the Arabidopsis Cer genes have been implicated in some aspects of alkane biosynthesis (Aarts, M. G., Keijzer, C. J., Stiekema, W. J., & Pereira, A. (1995) The Plant cell 7, 2115-2127). The genes encoding the enzymes that catalyze the key step of alkane biosynthesis—the conversion of fatty acids, fatty aldehydes or fatty alcohols to alkanes—are unknown.

Numerous organisms utilize alkanes and alkane response elements have been found in yeast and bacteria (Panke, S., Meyer, A., Huber, C. M., Witholt, B., & Wubbolts, M. G. (1999) Applied and environmental microbiology 65, 2324-2332; Souza, A. E., Myler, P. J., & Stuart, K. D. (1993) Gene 137, 349-350.). Alkane utilization pathways generally consist of an inducible promoter that includes an alkane response element (ARE) and drives transcription of one or more alkane utilization genes, and a transcriptional activator protein. Upon binding of a specific alkane, the transcriptional activator initiates transcription of the inducible promoter.

The best studied examples of alkane utilization pathways are the AREs from Pseudomonas putida mt-2 and Acinetobacter spp. The Pseudomonas ARE consists of the alkS transcriptional activator and the alkB promoter and responds to C6 to C10 n-alkanes. The Pseudomonas putida ARE has been used in E. coli to detect alkanes in the environment (Sticher, P., Jaspers, M. C., Stemmler, K., Harms, H., Zehnder, A. J., & van der Meer, J. R. (1997) Applied and environmental microbiology 63, 4053-4060). In that study, an E. coli strain was constructed that expressed the AlkS gene, and also carried a reporter gene under the control of the AlkB promoter. This E. coli plus Pseudomonas ARE responded to middle-length alkanes present in the environment. This recombinant cell responded to alkanes only, and only responded to environmentally provided alkanes.

The alkB promoter from Pseudomonas putida has also been used to express heterologous genes in E. coli and Pseudomonas (Smits, T. H., Seeger, M. A., Witholt, B., & van Beilen, J. B. (2001) Plasmid 46, 16-24). Again, the recombinant cells were shown to respond to externally provided n-alkanes only.

Three AREs have been described in Acinetobacter (Ratajczak, A., Geissdorfer, W., & Hillen, W. (1998) Journal of bacteriology 180, 5822-5827; Tani, A., Ishige, T., Sakai, Y., & Kato, N. (2001) Journal of bacteriology 183, 1819-1823.), Acinetobacter AREs consist of the alkR transcriptional activators and the alkM or alkB promoters. They respond to n-alkanes of different chain length, e.g., strain ADP1 responds to C7 to C18 and strain M1 responds to C16-C22 and >C22, respectively. To date, the Acinetobacter ARE has not been used in a heterologous cell. In addition, no ARE has been used to detect alkanes generated by enzymatic processes or to identify alkane biosynthesis and/or transport genes.

### SUMMARY OF THE INVENTION

Disclosed herein are methods and screening cells for identifying hydrocarbon pathway genes. Accordingly, one aspect of the invention is a method for identifying a hydrocarbon, e.g., alkane, pathway gene by expressing at least one candidate gene in a screening cell, e.g., E. coli, having a hydrocarbon responsive transcriptional activator, e.g., alkR and a reporter gene, e.g., GFP, driven by a hydrocarbon response element promoter, e.g., alkM, wherein the reporter gene is expressed in response to a hydrocarbon, e.g., an alkane; then detecting expression of the reporter gene; and identifying the candidate gene as a hydrocarbon pathway gene if the reporter gene is expressed in the screening cell.

In various embodiments, the hydrocarbon pathway gene is a hydrocarbon response gene or a hydrocarbon biosynthesis gene, or a hydrocarbon transport gene. In some embodiments, the method is used to screen a library, and the method includes transforming a population of the screening cells with a library comprising a plurality of candidate genes. The candidate genes can be from a prokaryotic organism, e.g., from Vibrio furnissii M1. The candidate genes can be from a eukaryotic organism, e.g., from Arabidopsis thaliana.

The screening cell is, for example, Escherichia coli, Acinetobacter species, or a Saccharomyces species. In a preferred embodiment, the screening cell is Escherichia coli. In some embodiment, the screening cell further additionally includes at least one hydrocarbon response gene, a hydrocarbon biosynthesis gene or a hydrocarbon transport gene.

In some embodiments, the hydrocarbon responsive transcriptional activator and the hydrocarbon response element promoter are from Pseudomonas putida mt-2 or Acinetobacter. In one variation, the reporter gene is GFP.

In some embodiments, screening cell responds to a hydrocarbon produced by the screening cell. Additionally, the screening cell responds to a hydrocarbon longer than C10.

In addition, the invention provides a screening cell comprising an Acinetobacter hydrocarbon responsive transcriptional activator and a reporter gene driven by an Acinetobacter hydrocarbon response element promoter wherein the reporter gene is expressed in response to a hydrocarbon. In some embodiments, the screening cell additionally includes a candidate gene, e.g., a hydrocarbon response gene, a hydrocarbon biosynthesis gene or a hydrocarbon transport gene. The candidate gene is from, e.g., Vibrio fumissii M1. In one variation, the screening cell is E. coli. The screening cell can include a hydrocarbon response gene or a hydrocarbon biosynthesis gene or a hydrocarbon transport gene. The reporter gene is, e.g., GFP.

In some embodiments, the screening cell responds to a hydrocarbon synthesized by the screening cell and/or responds to a hydrocarbon longer than C10.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a graph depicting induction in WH405 of various alkanes and alkenes. Note that heptadecene or 1-nonadecene at 50 mM resulted in cloudy culture medium; the miller units reported for these samples are therefore underestimates of the true values.

### DETAILED DESCRIPTION OF THE INVENTION

### Advantages and Utility

Briefly, and as described in more detail below, described herein are methods and screening cells for identification of hydrocarbon pathway genes. The methods utilize the alkane response elements from alkane responsive organisms, e.g., Acinetobacter, to drive expression of a reporter gene in a screening cell in response to hydrocarbons produced by the screening cell due to the co-expression of at least one hydrocarbon pathway genes.

Several features of the current approach should be noted. The method allows for intracellular detection of hydrocarbons produced within a transformed screening cell; the method does not require that the hydrocarbon be externally provided. In addition, the method allows detection of biosynthetic hydrocarbons having chain lengths that are advantageously longer than those previously shown to be detected using engineered reporting cells. The method also allows for detection of non-alkane hydrocarbons, e.g., alkenes and/or olefins and/or alcohols.

In some embodiments, the method uses the Acinetobacter ARE in a heterologous cell, e.g., E. coli, which has not been previously described.

As described herein, the invention is useful for identification of hydrocarbon biosynthesis genes, e.g., via screening libraries from hydrocarbon biosynthesizing species. The hydrocarbon biosynthesis genes are useful for development of hydrocarbon producing microorganisms and development of renewable sources of hydrocarbons.

The method is suited for enriching and screening hydrocarbon biosynthesis genes in metagenomic libraries and therefore could provide access to numerous novel alkane biosynthetic pathways from uncultured microorganisms in the future. This is crucial for discovery of hydrocarbon biosynthesis genes because the majority of microorganisms can not be cultivated and the ability to synthesize hydrocarbons may be widespread among uncultured microorganisms.

The methods described here can be used to clone and identify novel hydrocarbon biosynthetic genes without having prior knowledge of the molecular structure of these genes or related genes. This is important, because alkane biosynthesis genes have nor been studied on the molecular level. Therefore, methods that rely on prior knowledge of related genes (e.g. PCR with degenerate primers or DNA hybridization with heterologous probes) can not be applied. The alternative use of gene knock-out strategies to identify alkane biosynthesis genes (e.g. transposon mutagenesis) may also be flawed, because these pathways could be redundant in an alkane producer strain. Furthermore, some producer strains may not be amenable to genetic methods and applying more conventional mutagenesis strategies (e.g. nitrosoguanidines or UV) can be time and labor intensive.

The use of alkane response elements (AREs) coupled to GFP (or any other reporter gene) as described herein is a sensitive and fast method to identify hydrocarbon synthesizing and/or transporting and/or response genes. In addition, the method can be used for the optimization of such genes once they are cloned. The ability to screen for or optimize biosynthetic pathways for hydrocarbons with certain chain length is important, because hydrocarbons differ in their physical properties depending on the chain length, and therefore alkanes with different chain length may be used for different applications.

### Definitions

Terms used in the claims and specification are defined as set forth below unless otherwise specified.

“Accession numbers” are as derived from the NCBI database (National Center for Biotechnology Information) maintained by the National Institutes of Health, USA. The accession numbers are as provided in the database on Apr. 23, 2007.

“Hydrocarbon pathway gene” refers to a gene that plays a role in hydrocarbon biosynthesis including but not limited to synthesis (e.g., enzymes), transport (e.g., a receptor), or responsiveness (e.g., an enhancer).

“Candidate gene” refers to a potential hydrocarbon pathway gene. In some embodiments, the candidate gene is a library of candidate genes, e.g., a nucleic acid library (cDNA or genomic) derived from an alkane biosynthesis species.

“Screening cell” refers to a cell or population of cells having an ARE that expresses a reporter gene in response to a hydrocarbon. The terms screening cell and screening strain are used interchangeably.

“Polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

“Nucleic acid” refers to a ribose nucleic acid (RNA) or deoxyribose nucleic acid (DNA) polymer, or analog thereof. e.g., a nucleotide polymer comprising modifications of the nucleotides, a peptide nucleic acid, or the like. In certain applications, the nucleic acid can be a polymer including both RNA and DNA subunits. A nucleic acid can be, e.g., a chromosome or chromosomal segment, a vector (e.g., an expression vector), a naked DNA or RNA polymer, the product of a polymerase chain reaction (PCR), an oligonucleotide, a probe, etc.

“Operably linked” refers to linkage of a promoter to a nucleic acid sequence such that the promoter mediates/controls transcription of the nucleic acid sequence.

“Reporter gene” refers to gene or cDNA that expresses a product that is detectable by spectroscopic, photochemical, biochemical, enzymatic, immunochemical, electrical, optical or chemical means. Useful reporter genes in this regard include, but are not limited to fluorescent proteins, enzymes, and the like.

“Percent identity” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

“Heterologous nucleic acid” as used herein, refers to a nucleic acid wherein at least one of the following is true: (a) the nucleic acid is foreign (“exogenous”) to (i.e., not naturally found in) a given host microorganism or host cell; (b) the nucleic acid comprises a nucleotide sequence that is naturally found in (e.g., is “endogenous to”) a given host microorganism or host cell (e.g., the nucleic acid comprises a nucleotide sequence endogenous to the host microorganism or host cell); however, in the context of a heterologous nucleic acid, the same nucleotide sequence as found endogenously is produced in an unnatural (e.g., greater than expected or greater than naturally found) amount in the cell, or a nucleic acid comprising a nucleotide sequence that differs in sequence from the endogenous nucleotide sequence but encodes the same protein (having the same or substantially the same amino acid sequence) as found endogenously is produced in an unnatural (e.g., greater than expected or greater than naturally found) amount in the cell; (c) the nucleic acid comprises two or more nucleotide sequences that are not found in the same relationship to each other in nature, e.g., the nucleic acid is recombinant.

“Gene product” refers to a nucleic acid whose presence, absence, quantity, or nucleic acid sequence is indicative of a presence, absence, quantity, or nucleic acid composition of the gene. Gene products thus include, but are not limited to, an mRNA transcript, a cDNA reverse transcribed from an mRNA, an RNA transcribed from that cDNA, a DNA amplified from the cDNA, an RNA transcribed from the amplified DNA or subsequences of any of these nucleic acids. Polypeptides expressed by the gene or subsequences thereof are also gene products. The particular type of gene product will be evident from the context of the usage of the term.

It must be noted that, as used in the specification and the appended claims, the singular forms “a,” “an” and “the” include plural referents unless the context clearly dictates otherwise. Thus, for example, reference to “the screening cell” includes a population of screening cells.

Methods and Screening Cells of the Invention

Described herein is a method of screening for hydrocarbon pathway genes using a screening cell having an ARE operatively linked to a reporter gene. Candidate genes are introduced into the screening cell and identified as hydrocarbon pathway genes if the reporter gene is expressed.

The screening cell strain is developed as follows. The ARE from an alkane responsive strain, e.g., Acinetobacter sp. ADP1, is cloned such that the transcriptional activator, e.g. alkR or alkS, is adjacent to the promoter region of the alkane inducible gene, e.g. alkB or alkM. The promoter is operatively lined to a reporter gene, e.g., green fluorescent protein, e.g., GFP. The ARE-reporter gene containing vector is then transformed into a suitable cell type, e.g., E. coli. The ARE-reporter gene can be used as an autonomously replicating vector or the ARE-reporter gene cassette from the vector may be integrated into the screening cell chromosome by, e.g., homologous recombination. The resulting screening cell can be further manipulated to allow an improved flux of hydrocarbon precursors and/or to contain a partial hydrocarbon biosynthetic pathway. For example, the screening cell can include a hydrocarbon transport gene, or a hydrocarbon biosynthesis gene.

A candidate gene is introduced into the screening cell and identified as a hydrocarbon pathway gene if expression of the reporter gene is detected. In some embodiments, libraries of candidate genes are screened, e.g., metagenomic or genomic or cDNA libraries derived from hydrocarbon synthesizing species are screened for hydrocarbon pathway genes. The ability of the candidate gene to play a role in hydrocarbon biosynthesis (e.g., synthesis, transport, or response) is determined by the ability of the candidate gene to induce expression of the reporter gene.

Inducing Hydrocarbons

The screening cell responds to the presence of inducing hydrocarbons, e.g., expresses the reporter gene when hydrocarbons are detected by the screening cell. The inducing hydrocarbon can be in the environment. Alternatively, when used in the methods of the invention to screen candidate genes, the inducing hydrocarbons are present as the result of a hydrocarbon pathway gene present in the cell.

The selection of the ARE determines the length of the inducing hydrocarbon. In one embodiment, the screening cell responds to hydrocarbons of length C5 to C25. In another embodiment, the screening cell responds to hydrocarbons of length C7 to C22. In another embodiment, the screening cell responds to hydrocarbons of length C10-C15. In another embodiment, the screening cell responds to hydrocarbons of length >C25. For example, if the Acinetobacter ADP1 ARE is used, inducing hydrocarbons are of length C7 to C18. Genetic modification of an ARE can be used to modify the response of the screening cell, e.g., to modify the length of the inducing hydrocarbon.

The selection of the ARE also determines the bond composition of the inducing hydrocarbon. In one embodiment, the inducing hydrocarbon is an n-alkane. However, in other embodiments, the screening cell responds to other, non-alkane hydrocarbons. It is an advantage of the invention that non-alkanes can be used as inducing hydrocarbons, and that hydrocarbon pathway genes involved in synthesis, transport, and response to non-alkanes can be identified. Non-alkane inducing hydrocarbons includes but are not limited to alkenes and iso-alkenes (e.g., 1-hexadecene, 1-heptadecene, 1-heptadecene, 1-octadecene, 1-nonadecene, 1-eicosene, 9-cis-heneicosene and 9-cis tricosene) iso-alkenes, olefins, and the like.

Alkane Response Elements

The screening cell includes an alkane response element, e.g., an ARE operatively linked to a reporter gene. As described herein, an ARE includes an inducible promoter and a transcriptional activator gene. The ARE can be derived from any microorganism that possesses an inducible alkane utilization pathway, e.g. Acinetobacter baylyi ADP1, Acinetobacter sp. M1, Pseudomonas putida, Pseudomonas fluorescens, Pseudomonas aeruginosa, Rhodococcus erythropolis, Rhodococcus sp., Alcanivorax borkumensis, Gordonia sp., Mycobacterium tuberculosis, Prauserella rugosa, Burkholderia cepacia, Candida maltosa, Candida tropicalis, and Yarrowia lipolytica. For a recent review se: Beilen et al., Oil & Gas Science Technology, 2003, vol. 58, 427-440, which is herein incorporated by reference.

The nucleic acid sequences of the AREs used can be identical to those disclosed in the art and readily found on public databases, e.g., GenBank. Variants of the sequences can also be used, as long as the ARE accomplishes the same function, e.g., enables the screening cell to respond to the inducing hydrocarbon. For example, variant ARE sequences can be used that include modifications for optimized codon usage. Alternatively, variant ARE sequences can be used that modify the length or bond composition of the inducing hydrocarbon. The sequence of any known ARE can be altered in various ways known in the art to generate targeted changes in the amino acid sequence of the encoded transcription factor. The sequence changes may be substitutions, insertions or deletions. In addition, one or more nucleotide sequence differences can be introduced that result in conservative amino acid changes in the encoded protein.

In some embodiments, the ARE sequence is at least 90% identical to a previously disclosed sequences. In other embodiments, the ARE sequence is at least 91%, 92%, 93%, 94%, 95%, 96%, 97%, 98%, or at least 99% identical to the previously disclosed sequence.

In one embodiment, the ARE used is that from Acinetobacter baylyi ADP1 alkR/M locus (GenBank accession AJ002316, 7 Nov. 1997) (SEQ ID NO: 1), or Acinetobacter sp. M1 alkRb/Mb locus (GenBank accession AB049411, 27 Sep. 2000) (SEQ ID NO: 6) or Acinetobacter sp. M1 alkRa/Ma locus (Genbank accession AB049410, 27 Sep. 2000) (SEQ ID NO: 11). Depending on the application, various lengths of alkM coding sequence can be included in the ARE operatively linked to the reporter gene, e.g., at least 102 bases, at least 48 bases, or at least 3 bases of the alkM coding sequence. Exemplary sequences are disclosed in the sequence listing as described below.

Reporter Genes

The screening strain includes a reporter gene operably linked to an ARE promoter. In one embodiment, the reporter gene is GFP. One of skill will understand that any reporter genes can be used, e.g., β-galactosidase (lacZ), other florescent proteins (e.g. red fluorescent protein, DsRed), luciferase (luxAB), peroxidases, selectable antibiotic resistance genes (e.g., β lactamase, bla; aminoglycoside 3′-phosphotransferase, aph; chloramphenicol acetyltransferase, cat) and the like.

The mode for detection of expression depends on the reporter gene being used. In one embodiment, the reporter gene is GFP, and detection of expression is monitored by detecting or measuring GFP fluorescence (Crameri, A., Whitehorn, E. A., Tate, E., & Stemmer, W. P. (1996) Nature biotechnology 14, 315-319). E. coli expressing GFP can be screened on agar plates, in liquid culture or on single cell level by fluorescent activated cell sorting (FACS). GFPs with different fluorescent behavior and stability can be used depending on the application (Li, X., Zhao, X., Fang, Y., Jiang, X., Duong, T., Fan, C., Huang, C. C., & Kain, S. R. (1998) The Journal of biological chemistry 273, 34970-34975).

Sources of Candidate Genes

Using the methods of the invention, candidate genes are screened to identify hydrocarbon pathway genes, e.g., genes playing a role in hydrocarbon biosynthesis, transport, and response. Sources of candidate genes include any species that synthesizes hydrocarbons. Exemplary species are listed in Table 1 and Table 2 below. The method can be also used to enrich, identify and clone hydrocarbon biosynthesis genes from nucleic acid extracted from environmental samples, e.g. soil, seawater, sewage sludge, sea animals etc. For that purpose, metagenomic libraries can be constructed from such environments and introduced into a screening cell described herein.

TABLE 1
Hydrocarbon producing prokaryotes (examples)

| Strain | ATCC # or reference |
|---|---|
| Micrococcus luteus | ATCC 272 |
| Micrococcus luteus | ATCC 381 |
| Micrococcus luteus | ATCC 398 |
| Micrococcus sp. | ATCC 401 |
| Micrococcus roseus | ATCC 412 |
| Micrococcus roseus | ATCC 416 |
| Micrococcus roseus | ATCC 516 |
| Micrococcus sp. | ATCC 533 |
| Micrococcus luteus | ATCC 540 |
| Micrococcus luteus | ATCC 4698 |
| Micrococcus luteus | ATCC 7468 |
| Micrococcus luteus | ATCC 27141 |
| Jeotgalicoccus sp. | ATCC 8456 |
| Stenotrophomonas maltophilia | ATCC 17674 |
| Stenotrophomonas maltophilia | ATCC 17679 |
| Stenotrophomonas maltophilia | ATCC 17445 |
| Stenotrophomonas maltophilia | ATCC 17666 |
| Desulfovibrio desulfuricans | ATCC 29577 |
| Vibrio furnissii M1 | (1) |
| Clostridium pasteurianum | (2) |
| Anacystis (Synechococcus) nidulans | (3) |
| Nostoc muscorum | (3) |
| Cocochloris elabens | (3) |
| Chromatium sp. | (4) |

(1) Park, 2005, J. Bact., vol. 187, 1426-1429
(2) Bagaeva and Zinurova, 2004, Biochem (Moscow), vol. 69, 427-428
(3) Winters et al., 1969, Science, vol. 163, 467-468
(4) Jones and Young, 1970, Arch. Microbiol., vol. 70, 82-88

TABLE 2
Hydrocarbon producing eukaryotes (examples)

| Organism | ATCC # or reference |
|---|---|
| Cladosporium resinae | ATCC 22711 |
| Saccharomycodes ludwigii | ATCC 11311 |
| Saccharomyces cerevisiae | (5) |
| Botyrococcus braunii | (6) |
| Musca domestica | (7) |
| Arabidopsis thaliana | (8) |
| Pisum sativum | (9) |
| Podiceps nigricollis | (10) |

(5) Baraud et al., 1967, Compt. Rend. Acad. Aci. Paris, vol. 265, 83-85
(6) Dennis and Kolattukudy, 1992, PNAS, vol. 89, 5306-5310
(7) Reed et al., 1994, PNAS, vol. 91, 10000-10004
(8) Aarts et al., 1995, Plant Cell, vol. 7, 2115-2127
(9) Schneider and Kolattukudy, 2000, Arch. Biochem. Biophys., vol. 377, 341-349
(10) Cheesborough and Kolattukudy, 1988, J. Biol. Chem., vol 263, 2738-2743

Vectors

In many embodiments, the ARE-reporter gene construct in the screening cell is located on an expression vector. Similarly, in many embodiments, the candidate hydrocarbon pathway genes are present in expression vectors. Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and Acinetobacter). Thus, for example, a nucleic acid encoding a hydrocarbon pathway gene product(s) is included in any one of a variety of expression vectors for expressing the hydrocarbon pathway gene product(s). Such vectors include chromosomal, nonchromosomal and synthetic DNA sequences.

Numerous suitable expression vectors are known to those of skill in the art, and many are commercially available. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

Screening Cells

One of skill in the art will appreciate that any number of microorganisms can be used to generate the screening cell, including but limited not Escherichia coli strains, Acinetobacter spp., Saccharomyces spp., or any other microorganism, in which alkane response elements can be expressed, e.g., in which hydrocarbon-induced reporter gene expression can occur.

In one embodiment, E. coli is used to create the screening cell. An Acinetobacter ADP1 ARE:GFP construct is created and transformed into E. coli. The resulting screening strain expresses GFP in the presence of hydrocarbons. The hydrocarbon is provided externally, or, alternatively, the hydrocarbon is produced as the result of a co-transformed hydrocarbon biosynthesis gene.

In other embodiments, Acinetobacter is used as the starting cell to create a screening cell. Homologous recombination is used to create an insertion deletion in the Acinetobacter genome that replaces alkM, which encodes an alkane hydroxylase essential for alkane utilization, with a GFP gene under control of the alkM promoter. This strain cannot degrade alkanes but takes up and tolerates high concentrations of alkanes, which is an inherent feature of Acinetobacter. This Acinetobacter screening strain can respond to the presence of hydrocarbons by expressing GFP. A genomic library is created in a cosmid vector that can be stably replicated in Acinetobacter. The library is introduced into the Acinetobacter screening strain and screened for expression of GFP. Clones that contain functional genes involved in hydrocarbon biosynthesis will express GFP

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Expression of GFP Using Acinetobacter AREs in E. coli

Hydrocarbon induced expression of the reporter gene GFP in E. coli is demonstrated as follows. Briefly, reporter gene constructs are constructed using the ARE from Acinetobacter baylyi ADP1 alkR/M locus (GenBank accession AJ002316, 7 Nov. 1997) (SEQ ID NO:1), Acinetobacter sp. M1 alkRb/Mb locus (GenBank accession AB049411, 27 Sep. 2000) (SEQ ID NO:6) or Acinetobacter sp. M1 alkRa/Ma locus (Genbank accession AB049410, 27 Sep. 2000) (SEQ ID NO:11). Various ARE constructs are amplified from Acinetobacter genomic DNA to include restriction sites amenable to cloning. The isolated ARE construct fragments are ligated into an appropriate vector together with a nucleic acid fragment containing the GFP gene. The resulting vectors are transformed into E. coli and monitored for hydrocarbon induced expression of GFP.

Three different ARE constructs are isolated from each Acinetobacter species genomic DNA. Each construct include the coding sequences for the transcriptional activator, the intergenic region including the hydrocarbon inducible promoter, and differing length of alkM coding sequences, e.g., 102 bases, 48 bases, or 3 bases. Exemplary sequences are disclosed in the sequence listing as described in the following table:

| SEQ | | Included alkM | |
|---|---|---|---|
| ID NO: | Species | sequence | |
| 2 | Acinetobacter baylyi ADP1 alkR/M locus | 102 | bases |
| 3 | Acinetobacter baylyi ADP1 alkR/M locus | 48 | bases |
| 4 | Acinetobacter baylyi ADP1 alkR/M locus | 3 | bases |
| 7 | Acinetobacter sp. M1b alkRb/Mb locus | 102 | bases |
| 8 | Acinetobacter sp. M1b alkRb/Mb locus | 48 | bases |
| 9 | Acinetobacter sp. M1b alkRb/Mb locus | 3 | bases |
| 12 | Acinetobacter sp. M1a alkRa/Ma locus | 102 | bases |
| 13 | Acinetobacter sp. M1a alkRa/Ma locus | 48 | bases |
| 14 | Acinetobacter sp. M1a alkRa/Ma locus | 3 | bases |

The ARE constructs are PCR amplified from genomic DNA. All ARE forward primers specify HindIII and XhoI cloning sites. ADP1 and M1a reverse primers specify NcoI cloning sites. M1b reverse primers specify NdeI cloning sites. Reverse primers for amplification of DNA sequences that contain partial alkM sequence (e.g., SEQ ID NO: 2, 3) also specify ribosome binding sites for the reporter gene. Exemplary PCR primers are as follows:

| | | | |
|---|---|---|---|
| SEQ | | | |
| ID | Primer | | |
| NO: | name | Amplified Target | DNA Sequence (5′-3′) |

| | | | | |
|---|---|---|---|---|
| 15 | ADP1_F | ADP1 | forward | TTTTATTTCAAAGTCAAAGACTCGA |
| | | SEQ ID NO: 2, 3, 4 | | GAAGCTTGGG |
| 16 | ADP1_R1 | ADP1/alkM-102 | reverse | CATGCCATGGTATATCTCCTTAACT |
| | | SEQ ID NO: 2 | | AATCATCCATAAGTAAC |
| 17 | ADP1_R2 | ADP1/alkM-48 | reverse | CATGCCATGGTATATCTCCTTAATT |
| | | SEQ ID NO: 3 | | GATCACTTCTTCAAAGT |
| 18 | ADP1_R3 | ADP1/alkM-3 | reverse | CATGCCATGGTGAATCCTTTCTTGT |
| | | SEQ ID NO: 4 | | |
| 19 | M1b_F | M1b | forward | TCTTTAATCAGTTCGAGGAACTCGA |
| | | SEQ ID NO: 7, 8, 9 | | GAAGCTTGGG |
| 20 | M1b_R1 | M1b/alkM1b-102 | reverse | GAATTCCCATATGTATATCTCCTTA |
| | | SEQ ID NO: 7 | | ACCCATTGCAATGGTCGGTA |
| 21 | M1b_R2 | M1b/alkM1b-48 | reverse | GAATTCCCATATGTATATCTCCTTA |
| | | SEQ ID NO: 8 | | ATCCTTAAATGTTGTTGTCG |
| 22 | M1b_R3 | M1b/alkM1b-3 | reverse | GAATTCCCATATGCATTAGAAATTC |
| | | SEQ ID NO: 9 | | CT |
| 23 | M1a_F | M1b | forward | TTTTCTTTTCTGCTCAGTGACTCGA |
| | | SEQ ID NO: 12, 13, 14 | | GAAGCTTGGG |
| 24 | M1a_R1 | M1b/alkM1b-102 | reverse | CATGCCATGGTATATCTCCTTAGCT |
| | | SEQ ID NO: 12 | | AATGGCCCATAAATAAC |
| 25 | M1a-R2 | M1b/alkM1b-48 | reverse | CATGCCATGGTATATCTCCTTAGGC |
| | | SEQ ID NO: 13 | | TACTGGCGTAAGTTCTT |
| 26 | M1a_R3 | M1b/alkM1b-3 | reverse | CATGCCATGGAATCCATGTCTTTGT |
| | | SEQ ID NO: 14 | | |

In some embodiments, the native alkR sequence is replaced with an E. coli codon optimized sequence that translates to the same protein sequence. The codon optimized sequence and, in some embodiments, the native ARE, is created using custom-designed gene synthesis (http://www.dnatwopointo.com or http://www.codondevices.com). Exemplary codon optimized sequences are presented in the sequence listing as described in the following table:

| SEQ | | |
|---|---|---|
| ID NO: | Species | Description |
| 27 | Acinetobacter baylyi ADP1 alkR | Protein, GenBank accession |
| | | YP_046097, 29 Jun. 2004) |
| 5 | Acinetobacter baylyi ADP1 alkR | Codon optimized coding |
| | | sequence |
| 28 | Acinetobacter sp. M1 alkRb | Protein, GenBank accession |
| | | BAB33288, 27 Sep. 2000 |
| 10 | Acinetobacter sp. M1 alkRb | Codon optimized coding |
| | | sequence |
| 29 | Acinetobacter sp. M1 alkRa | Protein, GenBank accession |
| | | BAB33283, 27 Sep. 2000 |

A GFP reporter gene with a 5′ NdeI site was directly cloned from pJ1-FP2 (http://www.dnatwopointo.com). In order to specify a 5′ NcoI site, GFP was amplified from plasmid pJ1-FP2 using primer GFP_F_Nco (CATGCCATGGCGAGCAAAGGTGA) (SEQ ID NO:30) and GFP_R (specifies XhoI, HindIII and XbaI sites) (ACCTCTAGACTCGAGAAGCTTTT) (SEQ ID NO:31).

The ARE-GFP fusion cassettes are constructed as follows. ARE amplimers or plasmids containing ARE synthetic genes are digested with HindIII and NcoI (or NdeI, as appropriate). GFP amplimer is digested with NcoI and XbaI or GFP-containing pJ1-FP2 is digested with NdeI and XbaI. In a three-part ligation including one such digested ARE, one such digested GFP and HindIII/XbaI digested vector pAS4.22a, clones are created that contain ARE-GFP cassettes. pAS4.22a is a derivative of the low copy vector pACYC-Duet1 (Novagen) with an 1100 bp HpaI/BamHI deletion (to delete an inducible T7 promoter).

In some embodiments, the ARE::GFP cassettes from these constructs are moved into other vectors using standard cloning techniques.

The clones are transformed into E. coli BL21 (DE3) and grown on M9-minimal medium with either Glycerol or Glucose as carbon source supplemented with casamino acids (CAAs). Octane, decane, or hexadecane (10-60 mM final conc.) are added either immediately after inoculation or when the cultures have reached an OD₆₀₀ of 0.5-1.0. All experiments are done in screw cap tubes to prevent evaporation of volatile alkanes. Non-hydrocarbon controls are included to assay for effects on cell growth. GFP expression (under the control of AREs) is measured after 8, 24 and 32 h of growth at 37 C.

Results: ARE constructs that included only the first 3 bases of alkM (SEQ ID NO: 4, 9, and 14) were tested as described above. The ARE constructs included either native or codon optimized coding sequences for alkR. No hydrocarbon induced expression of GFP was observed.

AlkR was shown to be expressed as follows. The gene for alkR (native and codon optimized) from Acinetobacter ADP1 was cloned and operably linked under an IPTG inducible promoter. This construct was co-transformed with the respective ARE::GFP construct (see above) into E. coli BL21 (DE3). Experiments as described above were carried out with the exception that the cells were also induced with 1 mM of IPTG. No alkane-induced GFP expression was observed under these conditions. The expression of AlkR proteins in these experiments was analyzed by SDS PAGE, demonstrating that AlkR is expressed as a soluble protein (data not shown).

### Example 2

### Identification of Acinetobacter Genes Necessary for Screening Strain

As demonstrated by Example 1, Acinetobacter AlkR was expressed in E. coli, but the Acinetobacter ADP1 ARE (alkM-3) alone was insufficient for hydrocarbon induced expression of a reporter gene in E. coli. In some embodiments, at least one additional gene is necessary for to create the screening strain. This gene is identified by constructing an expression library from Acinetobacter genomic DNA and screening this library in E. coli harboring the ARE::GFP construct for GFP expression in the presence of hydrocarbons.

### Example 3

### Hydrocarbon Induction of an ARE:Reporter Gene in Acinetobacter WH405

Acinetobacter strain WH405, an ARE::lacZ derivative of strain ADP1 (Ratajczak et al 1998), was scraped from an overnight plate into LB and diluted to OD₆₀₀ 0.07 (the cells can also be from an overnight culture, as long as cells are diluted in fresh LB). Alkane and alkene induction of the ARE was tested by adding a number of alkenes at 5 and 50 mM to culture tubes, followed by addition of the diluted WH405 solution into tubes (5 mL). Tubes were incubated in a 37° C. shaker overnight. The β-galactosidase activity assay developed by Miller was used to determine the level of induction by the different hydrocarbon from the overnight cultures.

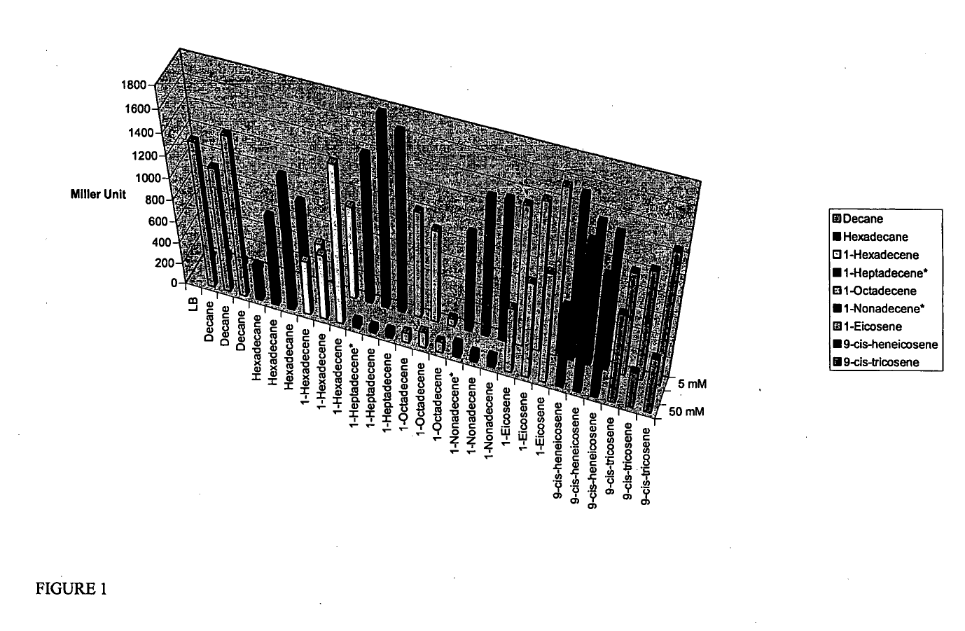

Results are shown in FIG. 1. In summary, decane and hexadecane as well as various alkenes (1-hexadecene, 1-heptadecene, 1-heptadecene, 1-octadecene, 1-nonadecene, 1-eicosene, 9-cis-heneicosene and 9-cis tricosene) induced the ARE in Acinetobacter WH405. In addition, octane (at 5 and 50 mM), but not hexane induces this ARE (data not shown).

It has not been previously shown that alkenes induce this ARE. It is known that the corresponding alcohol and acids of the same carbon length do not induce the ARE. Therefore, the Acinetobacter ARE is useful to screen for hydrocarbon biosynthesis genes wherein it is desirable to exclude genes for proteins that make alcohols or aldehydes. In addition, the Acinetobacter ARE is useful in a screening method wherein alcohol or aldehyde hydrocarbon precursors are supplied. Finally, the Acinetobacter ARE is useful in screening for hydrocarbons of C8 to at least C23. Therefore, this ARE is can be used in a method for identification of genes effecting alkene and alkane biosynthesis, such as those from Micrococcus, Stenotrophomonas, and Vibrio.

This is in contrast to the Pseudomonas ARE, which also responds to alcohols and ketones (Grund et al., J. Bact. 1975, vol. 123, p. 546-566 and has a different hydrocarbon specificity (C6-C12)

### Example 4

### Identification and Cloning of Novel Hydrocarbon Transporter Genes

A two strain system is developed for identification of hydrocarbon transporter genes, e.g., hydrocarbon exporters. The first strain, termed the producing strain, includes the genes necessary and sufficient for biosynthesis of intracellular hydrocarbons, but lacks the ability to release hydrocarbons to the medium. The second strain, termed the sensor strain, detects extracellular hydrocarbons, e.g., detects hydrocarbons in the medium. This sensor strain can be the screening strain described herein, e.g., E. coli or Acinetobacter carrying an ARE:reporter gene construct.

The first (producing) strain is transformed with a nucleic acid library, e.g., a genomic DNA or cDNA library, constructed from the DNA of any organism that is expected to possess an alkane transport gene. Individual clones of the transformed producing strain are cultured. If a transformed producing clone carries a hydrocarbon transporter gene, the cells secrete hydrocarbons into the medium. Hydrocarbons secreted by the producing strain clones are detected by the sensor strain either by growing both strains in the same medium, or, alternatively, by harvesting the medium from the producing strain and adding the medium to that of the sensor strain. If a transformed producing clone expresses a hydrocarbon transporter, the sensor strain expresses the reporter gene.

Alternatively, a sensor strain can be used that does not have an alkane induced reporter gene but can utilize alkanes as the sole source of carbon and energy. In the absence of any usable carbon source such a strain can not grow. If such a sensor strain is grown together (or comes into contact) with the E. coli expression library and if a clone secretes alkanes, the response strain will be able to grow without an added carbon source. This will allow identification of a clone expressing an alkane transporter gene.

### Example 5

### Optimization of Alkane Biosynthesis Pathway and Alkane Transporter Genes

The methods and screening strains are used to optimize components of the hydrocarbon biosynthetic machinery. For example, the methods and screening strains are used to screen for hydrocarbon biosynthesis proteins with improved properties (e.g. substrate turnover) or altered substrate specificity. General methods of mutagenesis are used to create libraries of mutant hydrocarbon biosynthesis or transporter genes. The mutagenized libraries are screened using the methods and screening strains described herein.

For example, to optimize alkane biosynthetic genes, ARE coupled to variants of GFP that are less stable in bacteria are used (11). Such a system allows the quantification of GFP expression and therefore can be used as a measure of the amounts of alkanes produced. By screening for elevated levels of GFP expression, improved alkane biosynthetic genes are identified.

Hydrocarbon biosynthetic genes with altered substrate specificity are also identified. A hydrocarbon pathway gene identified as described herein is mutagenized and screened for activity using different length of inducing hydrocarbons. For example, the gene that encodes a hydrocarbon pathway gene is identified using the Acinetobacter ADP1 ARE, e.g., the inducing hydrocarbon is of C14-C18 chain length. A library of mutant version of the gene is created and transformed into a different screening cell that includes a different ARE that responds to different length of inducing hydrocarbons. Transformed screening cell clones that express the reporter gene is response to inducing hydrocarbons are selected for further characterization.

The method can also be used in an analogous way to screen for alkane transporters with altered substrate specificity (i.e. transporters that efficiently transport alkanes with shorter or longer chain length). For this purpose an expression library of mutagenized alkane transporter genes would be screened in the two strain system described above under 3.2.2.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for identifying an hydrocarbon pathway gene comprising: expressing at least one candidate gene in a screening cell, said screening cell comprising an hydrocarbon responsive transcriptional activator and hydrocarbon response element promoter operably linked to a reporter gene wherein said reporter gene is expressed in response to an hydrocarbon; detecting expression of said reporter gene; and identifying the candidate gene as an hydrocarbon pathway gene if the reporter gene is expressed in the screening cell.

2. The method of claim 1, wherein said hydrocarbon pathway gene is an hydrocarbon response gene, an hydrocarbon biosynthesis gene, or an hydrocarbon transport gene.

3. The method of claim 1, further comprising transforming a population of said screening cells with a library comprising a plurality of candidate genes.

4. The method of claim 3, wherein said plurality of candidate genes are from a prokaryotic organism.

5. The method of claim 4, wherein said plurality of candidate genes are from Vibrio furnissii M1.

6. The method of claim 3, wherein said plurality of candidate genes are from a eukaryotic organism.

7. The method of claim 6, wherein said plurality of candidate genes are from Arabidopsis thaliana.

8. The method of claim 3, wherein said plurality of candidate genes are from a metagenomic library.

9. The method of claim 1, wherein said screening cell is Escherichia coli, a Acinetobacter species, or a Saccharomyces species.

10. The method of claim 1, wherein said screening cell further comprises at least one hydrocarbon response gene, hydrocarbon biosynthesis gene or hydrocarbon transport gene.

11. The method of claim 1, wherein said hydrocarbon responsive transcriptional activator and said hydrocarbon response element promoter are from Acinetobacter.

12. The method of claim 1, wherein said reporter gene is GFP

13. The method of claim 1, wherein said screening cell responds to a hydrocarbon produced by the screening cell or to a hydrocarbon longer than C10.

14. The method of claim 1, wherein said screening cell is E. coli or Acinetobacter, said hydrocarbon responsive transcriptional activator is Acinetobacter alkR, said reporter gene is GFP, said hydrocarbon response element promoter is Acinetobacter alkB or alkM; and expression is detected using FACs.

15. A screening cell comprising an Acinetobacter hydrocarbon responsive transcriptional activator and an Acinetobacter hydrocarbon response element promoter operably linked to a GFP gene, wherein said GFP gene is expressed in response to a hydrocarbon.

16. The screening cell of claim 15, further comprising a candidate gene.

17. The screening cell of claim 15, wherein said screening cell is E. coli.

18. The screening cell of claim 15, wherein said screening cell further comprises an hydrocarbon response gene or an hydrocarbon biosynthesis gene or an hydrocarbon transport gene.

19. The screening cell of claim 15, wherein said screening cell responds to a hydrocarbon synthesized by the screening cell or to a hydrocarbon longer than C10.

20. The screening cell of claim 15, wherein said screening cell is E. coli or Acinetobacter, said hydrocarbon responsive transcriptional activator is Acinetobacter alkR, said reporter gene is GFP, and said hydrocarbon response element promoter is Acinetobacter alkB or alkM.
